(19) United States
(12) Patent Application Publication
(10) Pub. No.: US 2018/0360923 A1
(43) Pub. Date: Dec. 20, 2018

(54) Oncological and Neurological Therapy based on ATM, CHK2 and p53 proteins for DNA correction of transcription errors

(71) Applicant: Pedro Brito da Silva Correia, Estoril (PT)

(72) Inventor: Pedro Brito da Silva Correia, Estoril (PT)

(21) Appl. No.: 15/626,132

(22) Filed: Jun. 18, 2017

(51) Int. Cl. A61K 38/45; A61K 38/17

(52) U.S. Cl. CPC A61K 38/45; A61K 38/1758; C12Y 207/11001

(57) ABSTRACT

Important diseases are caused by mutations in some genes of DNA, like cancer and several neurologic diseases. These mutations are corrected in healthy humans by a complex mechanism triggered by the proteins ATM, CHK2 and p53. These proteins cause apoptosis, whenever the transcription error in DNA is too large to be corrected.

This mechanism does not work if the genes encoding several proteins, mainly ATM, CHK2 and p53, are naturally mutated or are experimentally removed in rats using CRISPR or similar processes.

We claim the therapeutic use of the proteins ATM, CHK2 and p53 to activate the natural mechanism of DNA transcription correction.

These proteins are identical to those existing in healthy cells, and are already being produced for research or diagnosis purposes by monoclonal processes.

### 1. FIELD OF INVENTION

Oncology, Parkinson disease, Alzheimer disease, DNA transcription correction mechanism

### 2. BACKGROUND OF THE INVENTION

Diseases caused by virus, bacteria or other foreign bodies, which can be detected through antigens by the immunitarian system, are eliminated, only under the condition that the membranes of the limphatic cells B or fagocitic cells contain the protein with a partop which may be connected to the epitop of the corresponding antigen.

Other diseases are not associated with antigens, but caused by the transcription of some genes of DNA with mutations. In some cases the cells have the ability to correct the mutation or to cause apoptosis of the cell containing the mutated gene. If this mechanism does not work, the mutated genes produce proteins which originate diseases like
cancer: pre oncogenes RAS, WNT, MYC, ERK, TRKA, BCR-ABL, DHH, SHH, IHH, NURR1, LIF, VEGF, NGF, BDNF, NT3, NT4
Parkinson: genes LRRK2, ATN, producing alfa synuclein
Alzheimer: genes APP, PSEN1 and PSEN2 which cause beta amyloide production.

The discovery of the repair of DNA transcriptional errors was recognised as a major achievement by the award of the Nobel Prize for Chemistry in 2015 to Paul Modrich, Aziz Somar and Thomas Lindahl.

ATM, CHK2 and p53 trigger the correction mechanism of mutated genes in cells or cause apoptosis, whenever the transcription error is too large to be corrected.

ATM are the initials for Ataxia Telangiectasia. This is a disease where the protein ATM does not exist and the mutations of DNA are not corrected.

ATR is a complex of ATM and RAD3.

ATM is a kynase, a family of proteins which has the capacity to phosphorylate other proteins, and by this chemical reaction supply the energy necessary for the conversion of the initial protein in a protein with more energy. Phosphorylation is the enzymatic form to make chemical reactions without heating, as it is usually necessary for chemistry in the laboratory. The family of kynases are produced in mitochondria.

ATM has a molecular structure which allows the formation of a cycle which embraces and scans the DNA for anomalies, in a similar way as the PCNA which participates later in the repair.

ATM participates in 2 steps:
activates the protein complex MRN, which includes the proteins MRE11, RAD50, NBS1 (Nijmegen Breakage Syndrome). The proteins RAD50 and MRE11 cause the alignment of the two DNA chains during repair. Protein NBS1 participates together with telomerase in the repair of the telomeres, situated at the tops of each gene.
The activated complex MRN changes the conformation of ATM, which increases its affinity to the proteins CHK2 and p53 which are activated.

The protein ATM is produced by the gene ATM, which is located in the chromossome 11. ATM is a dimer, which is inactive in healthy cells. In the cells containing a mutation in DNA, ATM phosphorilates itself, which causes the dissociation in the two monomers. The monomers become activated and interact with the protein CHEK2 (chromossome 22), which following activate the protein p53.

A mutation in the gene CHEK2 producing the protein CHEK2, causes the impossibility of CHEK2 to participate in the repair mechanism of DNA and causes cancer or neurologic diseases caused by genetic disorders.

The protein p53 controls if the cells should have a DNA repaired or should suffer apoptosis, whenever the repair is not possible.

Oncologic cells do not have this protein or have a mutated form of it. These cells may have the gene p53, but it is not expressed into the corresponding protein.

As observed by Richard Peto, cancer is not proportional to the number of cells of the body. It was found in 2015 that elephants and whales have 10 to 20 genes p53, while humans have just one. As a consequence, elephants and whales have a very low incidence of cancer. In fact, if there is a mutation in one p53 gene, another identical gene produces the same protein to trigger the repair of the mutated genes.

The gene p53 is located in the chromossome 17. It produces 15 proteins designated by isomorphic, which participate in the repair of DNA.

There are humans with no gene p53 at all. They have a high incidence of cancer, which constitutes the síndrome of Li Fraumeni.

The first commercial gene therapy was approved in 2003 in China and is called Gendicine, but it was not approved in USA and EU. Gendicine introduces the gene p53 in the tumor cells using an adenovirus.

This therapy may have a side effect, which results from the fact that both healthy cells and some cells containing mutated DNA may be repaired. If cells containing mutated DNA are repaired in a way that no healthy cells are produced but other cells containing the mutations of the mother cell, the disease is not eliminated.

Therefore, healthy cells must have the proteins necessary for repairing mutations whenever they occur. If there are already too many mutations, the cells suffer apoptosis.

According to references 10 and 11, in response to DNA damage, cells activate the sensor kinases ATM, ATR and DNA-PK, that in turn phosphorylate multiple downstream substrates, including the effector kinases Chk1 and Chk2, resulting in cell-cycle checkpoint initiation and/or apoptosis

Of the four intervenient ATR, ATM, CHK1 or CHK2, CHK2 is the most commonly altered and approximately 50% of GBM patients with CHK2 alterations also carry defects in the p53 signaling pathway (such as TP53 mutation/loss or amplifications of MDM2/4, both well-known p53 inhibitors).

CHK2 is known to be required for the p53-dependent apoptotic response to radiation. In order to analyze the role of p53 in Chk2 mediated tumor suppression, the authors in reference 11 crossed Ntv-a Chk2^{-/-} mice with p53^{-/-} mice and noted that PDGF-induced gliomas arise with a similar latency in both genetic backgrounds. Furthermore, studies have been made suggesting that CHK2 and p53 are epistatic in the suppression of glioma formation. Epistasis is the need for a group genes to act and not one gene alone.

The G1/S checkpoint, which prevents cells from entering S phase, is predominantly regulated by p53 and is defective in ATM null cells. Despite its well-known function in ATM-dependent IR-induced p53 activation, the role of CHK2 in the G1/S checkpoint is still controversial.

CHK2 is required for the maintenance of the G2/M checkpoint in gliomas, but not for its activation, either in vitro or in vivo.

ATM directly phosphorylates p53 on Ser-15 and Thr-68 on CHK2, which, in turn, phosphorylates p53 on Ser-20, thereby helping to regulate the cell cycle and apoptosis. Thus, CHK2 works as both a transducer acting in the ATM-CHK2-p53 cascade and a candidate tumor suppressor. Indeed, CHK2 mutations are found in some hereditary malignancies, such as Li-Fraumeni Syndrome.

Oncological and Neurological Therapy based on ATM, CHK2 and p53 proteins for DNA correction of transcription errors

### 3. SPECIFICATION OF THE INVENTION

We found very interesting the therapeutic use of the proteins ATM, CHK2 and p53, which activate the mechanism of correction of transcription errors in DNA.

ATM, CHK2 and p53, are identical to those existing in healthy cells, and are already being produced for research or diagnosis purposes by monoclonal processes.

We consider that a protein therapy is better than a gene therapy for diseases caused by mutations in gene, by following two reasons:
Proteins introduced in the cells are removed after a certain time by mechanisms existing in the cell based on ubiquitin, while genes can hardly be removed by a natural way, not using laboratory processes like CRISPR. The permanence of artificially introduced genes may cause undesirable side effects.

The package of proteins to use in the therapy can be selected in order to include not only ATM, CHK2 and p53 but also some important proteins which are missing in the human.

Therefore, we concluded the advantage of the intravenous local injection of a plasma solution of the proteins ATM, CHK2 and p53 in order to supply the means for the activation of the repair mechanism of DNA.

### EXAMPLE

The hair of 5 Rats is removed and a melanoma is induced. They are treated with one weekly injection of a 1 ml solution containing plasma and 1 microgram ATM, 1 microgram of CHK2 and 1 microgram p53.

After 2 months the melanoma of these rats is compared with five rats which were not injected, and the reduction of the melanoma cells is checked.

1. We claim a therapeutic process using a plasma solution of the proteins ATM, CHK2 and p53 produced my well known monoclonal antibody technology, which is injected in the blood of patients suffering from any disease caused by gene mutations, like cancer, Parkinson's or Alzheimer diseases

2. In the process of claim 1 where the therapy is complemented with any protein found to be missing in the mechanism of gene repair in the individual patient

3. In the process of claim 1 where the patient is checked every month in order to follow the correct adaptation of the protein mix to the mutated genes repair

4. In the process of claim 1 where the quantity to be administered may vary between 1 and 10 microgram per treatment and per protein according to the weight of the person and the reaction to the first administered quantities.
